(19) United States
(12) Patent Application Publication
(10) Pub. No.: US 2024/0153589 A1
(43) Pub. Date: May 9, 2024

(54) SYSTEM AND METHOD FOR IMPROVING HIGH-PRECISION ION MOBILITY WORKFLOW

(71) Applicant: BiognoSYS AG, Schlieren (CH)

(72) Inventors: Tejas Paresh GANDHI, Lenzburg (CH); Lukas REITER, Thalwil (CH); Oliver BERNHARDT, Urdorf (CH)

(73) Assignee: BiognoSYS AG, Schlieren (CH)

(21) Appl. No.: 18/280,118

(22) PCT Filed: Feb. 11, 2022
(86) PCT No.: PCT/EP2022/053387

§ 371 Date: Sep. 1, 2023

Related U.S. Application Data
(60) Provisional application No. 63/156,971, filed on Mar. 5, 2021.

(51) Int. Cl. G16B 40/10; G01N 30/72; G01N 30/88

(52) U.S. Cl. CPC G16B 40/10; G01N 30/7233; G01N 30/88; G01N 2030/027

(57) ABSTRACT

Method for precursor identification from mass spectroscopic data as a function of mass to charge ratio, retention time as well as of ion mobility, using a database of reference precursor data for retrieval of a region of interest for at least three reference peptide precursors in the mass to charge ratio, the retention time as well as in the ion mobility dimension. In a first step for at least three reference precursors, from the database, said data is analysed in the precursor region of interest of mass to charge ratio, retention time as well as ion mobility dimension, and from that analysis empirically an adjusted center in the ion mobility dimension is determined and an ion mobility extraction width window is determined, and in a second step for the identification of further peptide precursors, said extraction width window is used.

### TECHNICAL FIELD

The present invention relates to the analysis of compounds in mass spectrometry and more particularly to instruments, and methods for polypeptide analysis.

### PRIOR ART

Targeted analysis of data-independent acquisition (DIA) data is a powerful mass spectrometric approach for comprehensive, reproducible, and precise proteome quantitation. It requires a spectral library which contains peptide precursor ions to be used for the targeted extraction in DIA data. This targeted extraction could be over multiple dimensions, such as retention time, ion mobility, etc. The existing methods do not speedily or efficiently analyze the data in multiple dimensions in order to generate quantification of all data (for as many peptides as possible). One way of achieving the targeted extraction is to use the full range of available data when searching for a given peptide precursor ion. However, this degrades the overall analysis due to increased occurrence of interferences in the data. Furthermore, it also leads to significantly longer analysis times as more data needs to be processed. Another way of achieving the targeted extraction is by using only the retention time dimension. However, this does not take into account the ion mobility dimension. Therefore, a system and method are needed that can achieve the targeted extraction in the ion mobility dimension in addition to the retention time dimension.

### SUMMARY OF THE INVENTION

The present implementation is fast and robust, and it analyses data in a targeted fashion using multiple layers of calibration, a suitable data structure for fast access, and fall back options. Mass spectrometer instruments use the ion mobility-based separation which requires a four-dimensional analysis for a compound in terms of its mass to charge ratio m/z, retention time RT, ion mobility IM, and intensity. Unlike the current solutions which make use of static user-predefined extraction width in ion mobility dimension (see e.g. Yu et al., 2020 in “Fast Quantitative Analysis of timsTOF PASEF Data with MSFragger and IonQuant”, Mol Cell Proteomics 19(9), 1575-1585), the present solution improves the extraction significantly by empirically determining a peptide precursor ion-specific ion mobility (IM) extraction width. This allows it to optimally adapt prediction precision depending on the quality of the underlying data. A person with ordinary skill in the art will understand that in addition to proteomics, this workflow can also be applicable to other mass spectrometer-based omics data, including but not limited to metabolomics.

The present invention determines position and extraction width in the ion mobility dimension for each precursor ion in the library. In one implementation, a model is created. The model, which can also be a machine learning model, is in the form of a segmented regression of predicted ion mobility versus empirically observed ion mobility. The model is iteratively refined till finalized. The final model can predict a) the location of the peptide in the ion mobility accounting for systematic shifts and thereby increasing accuracy, and b) tolerance in the form of extraction width which accounts for precision. For example, if the empirical ion mobility data is in good agreement with the library value then the model will use narrower extraction width.

Next, a novel module facilitates quick construction of mobilograms in the m/z—ion mobility dimension based on the extraction width determined in each iteration and also construction of extracted ion chromatograms (XIC) in the m/z—retention time dimension by summarizing the ion mobility dimension. This module is used for both MS1 level (consisting of precursor ion features) and MS2 level (consisting of fragment ions of precursor ions after the fragmentation process in the mass spec.). A person with ordinary skill in the art will understand that such a module can consist of data structure, algorithm, a program code or similar components.

More specifically, the present invention proposes the analytical methods as claimed in the appended claims.

We propose a method for the targeted peptide precursor identification from sample mass spectroscopic intensity data acquired as a function of mass to charge ratio (m/z), of retention time (RT) as well as of ion mobility (IM).

The method is using a database of reference peptide precursor data for retrieval of a region of interest for at least three reference peptide precursors in the mass to charge ratio (m/z), the retention time (RT) as well as in the ion mobility (IM) dimension.

According to the proposed method, for peptide precursor identification, scoring, quantification or a combination thereof from the sample mass spectroscopic intensity data,
1. in a first step for at least three reference peptide precursors, preferably for all reference peptide precursors from the database of reference peptide precursor data, said sample mass spectroscopic intensity data is analysed in the respective reference peptide precursor region of interest of mass to charge ratio (m/z), retention time (RT) as well as ion mobility (IM) dimension, and from that analysis empirically an adjusted center in the ion mobility dimension (IM) for each reference peptide precursor is determined and an ion mobility extraction width window in the ion mobility dimension (IM), preferably as a variable function of the ion mobility dimension (IM), is determined, and
2. in a second step for the identification of further peptide precursors from said sample mass spectroscopic intensity data, said empirically determined ion mobility extraction width window in the ion mobility dimension (IM), preferably as a variable function of the ion mobility dimension (IM) is used.

According to a first preferred embodiment of the proposed method, for the analysis in the first and/or the second step, for a given retention time (RT) the data are merged into a single array with three dimensions, mass to charge ratio (m/z) dimension, intensity dimension, and ion mobility (IM) index dimension, sorted by m/z. Preferably this single array is used for the analysis in the first as well as in the second step, since it provides for optimum access to the data for quick, robust and reliable data analysis.

More specifically, according to a preferred embodiment in said first step for each reference peptide precursor the analysis considers a range in the retention time (RT) dimension as a retention time (RT) window, preferably the full retention time (RT) dimension. For each retention time (RT) value in that retention time (RT) window, the method accesses said single array for that retention time (RT) for building a first ion trace for that reference peptide precursor.

This first ion trace for that reference peptide is built up in that the intensity values that fall within a mass to charge ratio (m/z) window of the corresponding reference peptide (as retrieved from the database or from previous runs) are summed up over the full range in the ion mobility dimension (IM) to a single data point for that retention time (RT) value, and these single data points as a function of the retention time (RT) value are put together to build said first ion trace in the retention time (RT) dimension.

In that first ion trace in the following peak detection is carried out to determine the apex retention time (RT) and preferably also the peak width in the retention time (RT) dimension for that reference peptide precursor.

According to yet another preferred embodiment, the building of the first ion trace and the determination of the apex retention time (RT) and preferably also the peak width in the retention time (RT) dimension for that reference peptide precursor is followed by extraction of a second ion trace at said trace apex retention time (RT) (so not over a window any more) by accessing said single array for that trace apex retention time (RT) for building a second ion trace for that reference peptide precursor. In the following the intensity values that fall within a mass to charge ratio (m/z) window of the corresponding reference peptide are extracted and represented as a function of the ion mobility dimension (IM), building together said second ion trace in the ion mobility dimension (IM).

This is then followed by peak detection in the second ion trace to determine the apex ion mobility (IM) value and the peak width for that reference peptide precursor.

The peak detection in the second ion trace can, as preferred, be followed by a numerical optimisation, preferably a non-linear or linear ion mobility (IM) regression using the apex ion mobility (IM) values and the peak widths of the at least three reference peptide precursors to determine said adjusted center in the ion mobility dimension (IM) for each reference peptide precursor and to determine said window in the ion mobility dimension (IM), preferably as a variable function of the ion mobility dimension (IM).

It is to be noted that the adjusted centre in the ion mobility dimension determined here is generally not the centre in the ion mobility dimension when just looking at that very reference peptide precursor in the data, but it is a centre which is determined based on the data of all the reference peptide precursors that are being analysed in this step and which represents the corresponding centre position according to the numerical optimisation. It therefore includes and compensates corresponding shifts.

According to yet another preferred embodiment, said first step is carried out for a first set of at least 3, preferably at least 5, or at least 7, or at least 10, or at least 100, or at least 1000 reference peptide precursors considering the full range in the retention time (RT) dimension as retention time (RT) window for building the first ion trace, and said first step is further preferably carried out a second time for a more refined calibration with a larger set of reference peptide precursors than the first time, preferably at least 10, more preferably at least 100, more preferably at least 1000, or at least 2000, or at least 5000 reference peptide precursors, and wherein a retention time (RT) window is used for building the first ion trace based on the peak width in the retention time (RT) dimensions determined in the first run of the first step.

Preferably, using said database of reference peptide precursor data for a region of interest for at least 5, or at least 7, or at least 10, or at least 100, or at least 500, or at least 1000 reference peptide precursors in the mass to charge ratio (m/z), the retention time (RT) as well as in the ion mobility (IM) dimension is retrieved for carrying out the first step. According to another preferred embodiment, in said second step after the determination of the precursors in the data, for each precursor the analysis considers a range in the retention time (RT) dimension as a retention time (RT) window, preferably determined based on an analysis of reference peptide precursors. For each retention time (RT) value in that retention time (RT) window, according to this embodiment in the second step the method accesses said single array for that retention time (RT) for building a first ion trace for that precursor. The first ion trace for that precursor is built up in that the intensity values that fall within a mass to charge ratio (m/z) window of the corresponding precursor are summed up over the full range in the ion mobility dimension (IM) to a single data point for that retention time (RT) value, and these single data points as a function of the retention time (RT) value are building together said first ion trace in the retention time (RT) dimension. Typically this is followed by peak detection in the first ion trace to determine the apex retention time (RT) and preferably also the peak width in the retention time (RT) dimension for that precursor.

In the context of this second step preferably the step of peak detection in the first ion trace to determine the apex retention time (RT) and preferably also the peak width in the retention time (RT) dimension for that precursor is followed by extraction of a second ion trace at said trace apex retention time (RT) by accessing said single array for that trace apex retention time (RT) for building a second ion trace for that precursor. Then typically the intensity values that fall within a mass to charge ratio (m/z) window of the corresponding precursor are extracted and represented as a function of the ion mobility dimension (IM), building together said second ion trace in the ion mobility dimension (IM). This is typically followed by peak detection in the second ion trace to determine the apex ion mobility (IM) value and the peak width for that precursor for scoring and/or identification of the precursor.

Preferably, in the first step also in the mass to charge ratio (m/z) dimension and/or in the retention time (RT) dimension an extraction width window in the respective dimension, preferably as a variable function of the respective dimension, is determined, and in the second step for the identification of further peptide precursors from said sample mass spectroscopic intensity data, said empirically determined extraction width window in the respective dimension as a variable function of the respective dimension is used. The sample mass spectroscopic intensity data can for example have been acquired as a function of mass to charge ratio (m/z), of retention time (RT) as well as of ion mobility (IM) are determined using an LC tandem mass spectroscopy method, preferably selected from the group of LC-MRM or LC-SWATH is used.

Liquid chromatography coupled to Mass Spectrometry (LC-MS) has now been used for many years in the proteomic community for the identification and quantification of peptides (and thus proteins) from complex sample mixtures. In proteomics, the analytes are typically peptides generated by tryptic digestion of protein samples. The commonly most used approaches are variants of the so called LC-MS/MS or “shotgun” MS approach that is based on the generation of fragment ions from precursor ions that are automatically selected based on the precursor ion profiles (data dependent analysis, DDA). The most mature technology is called selected Reaction Monitoring (SRM), frequently also referred to as multiple reaction monitoring (MRM). The targets for MRM experiments are defined on a rational basis and depend on the hypothesis to be tested in the experiment. Selected combinations of precursor ions and fragment ions (so called transitions, the set of transitions for one target precursor is called MRM assays) for these targets are programmed into a mass spectrometer, which then generates measurement data only for the defined targets. Another variant of targeted proteomics is data independent acquisition, and a more recently presented variant commonly called SWATH-MS approach. Here, the targeted aspect is introduced only on the data analysis level. Contrary to MRM, this approach does not require any preliminary method design prior to the sample injection. Since the LC-MS acquisition covers the complete analyte contents of a sample through the entire mass and retention time (RT) ranges the data can be mined a posteriori for any peptide/precursor of interest. Data is acquired in a data independent manner, on the complete mass range (e.g. 200-2000 Thomson) and through the entire chromatography, disregarding of the content of the sample. This is commonly achieved by stepping the selection window of the mass analyzer step by step through the complete mass range. In effect, this data acquisition method generates a complete fragment ion map for all the analytes present in the sample and relates the fragment ion spectra back to the precursor ion selection window in which the fragment ion spectra were acquired. This is achieved by widening the precursor isolation windows on the mass analyzer and thus accounting a priori for multiple precursors co-eluting and concomitantly participating to the fragmentation pattern recorded during the analysis. Such a precursor window is called a swath. The result is complex fragment ion spectra from multiple precursor fragmentations, that require a more challenging data analysis.

Unlike in shotgun proteomics, for the MRM and SWATH technology spectra are repeatedly recorded for the same analytes with a high time resolution. The high time resolution when compared to shotgun proteomics, together with the limited fragment ion information for MRM and the limited fragment ion to precursor ion association for SWATH, makes a completely new type of data analysis necessary. Since only a limited number of pre-defined analytes are being monitored, it is not necessary to make a shotgun proteomics type database search by comparing the spectra to a complete theoretical proteome. Instead, a number of scores have been described that are based on signal features such as shape, co-elution of transitions, and similarity of transition intensities to assay libraries.

Typically, the sample mass spectroscopic intensity data have been acquired as a function of mass to charge ratio (m/z), of retention time (RT) as well as of ion mobility (IM) from a biological sample in a data independent manner on the complete mass range, preferably 200-2000 Thomson, and through the entire chromatography, disregarding of the content of the sample.

At least one, preferably at least 2, or at least 5, or at least 7 reference peptide precursors for carrying out the first step can be based on a preferably corresponding number of proteins or peptides spiked into the sample to be analysed prior to analysis.

Furthermore the present invention relates to a method for the generation of a peptide precursor database, in particular for use in a method as described above, wherein at least one or preferably for the majority or for each peptide precursor in the database based on empirical measurements an associated ion mobility extraction width window is determined and stored in the database preferably determined from sample mass spectroscopic intensity data acquired as a function of mass to charge ratio (m/z), of retention time (RT) as well as of ion mobility (IM), and using a method as described above.

Last but not least the present invention relates to a computer program product, preferably on or comprising a tangible computer-readable storage medium whose contents include a program with instructions being executed on the processor so as to control a device for chemical analysis using a method according to any of the preceding claims.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows the apex of the signal of a peptide precursor ion in 3-dimensional space consisting of ion mobility, retention time, and intensity for a given m/z value;

FIG. 2 shows the data structure referred to as 3D IM Scan and its relationship with the raw data coming from the mass spectrometer;

FIG. 3 illustrates an extensive calibration process;

FIG. 4 illustrates the main analysis pipeline;

FIG. 5 illustrates a method to construct an ion trace using an example;

FIG. 6 illustrates a method to construct a mobilogram 503a using an example; and

FIG. 7 illustrates an optimal extraction width for each set of experimental data based on the performance of the instrument and quality of the library;

FIG. 8 illustrates an ion mobility calibration (Predicted vs. Empirical) on a sample data set; and

FIG. 9 illustrates an extraction width in ion mobility dimension on a sample data set.

### DESCRIPTION OF PREFERRED EMBODIMENTS

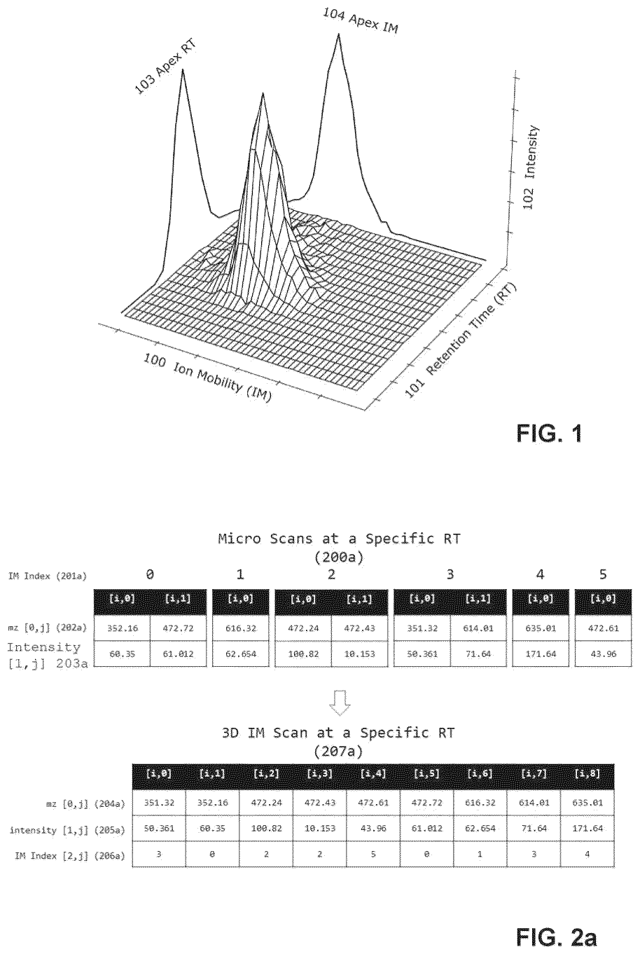

FIG. 1 illustrates a peptide precursor ion in 3-dimensional space consisting of ion mobility 100, retention time 101, and intensity 102 for a given m/z value. We also show the 2-dimensional projection of retention time (RT) vs. intensity, and ion mobility (IM) vs. intensity along with their respective apex points 103 and 104, respectively. These points103 and 104 are very important in targeted analysis as they predict the position of the peptide precursor in the corresponding dimensions.

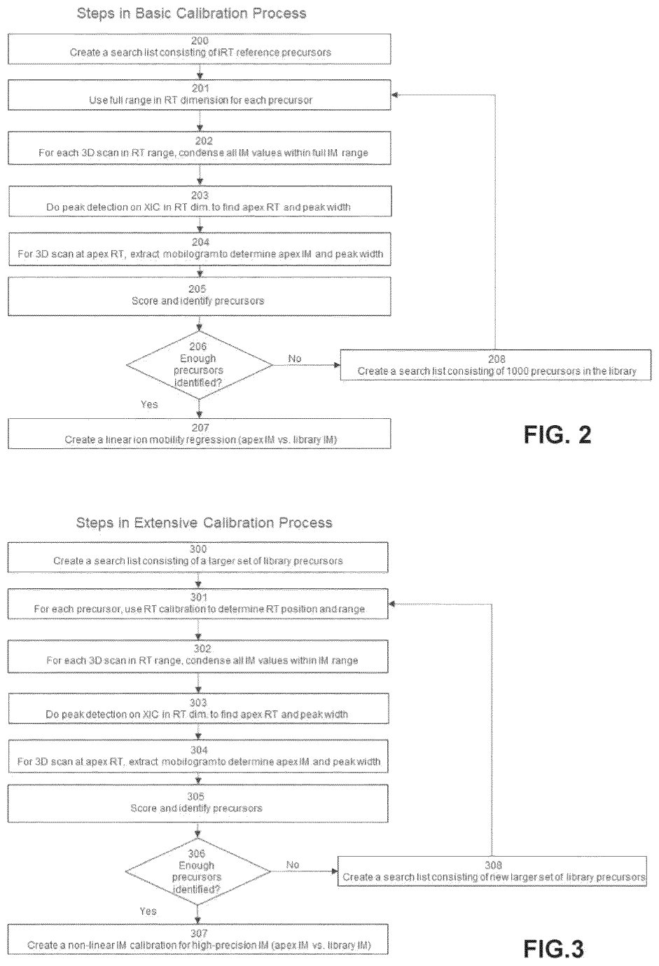

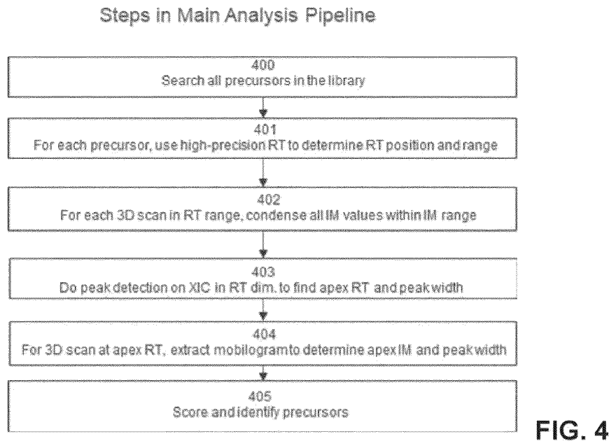

The high-precision ion mobility workflow can be illustrated using three implementations of the novel system and method: basic calibration (FIG. 2), extensive calibration (FIG. 3), and main analysis pipeline (FIG. 4).

But before describing the high-precision ion mobility workflow, it is important to describe the data structure that is used for encoding the ion mobility for quick access (FIG. 2a). A person with ordinary skill in the art will understand that the three implementations can be used individually or any combination with the other implementation.

FIG. 2a illustrates the data structure referred to as 3D IM Scan 207a and its relationship with the raw data coming from the mass spectrometer. In one implementation, at every given RT position, the mass spectrometer can separate the compounds by their ion mobility. The data representing a full IM scan is recorded in multiple micro scans 200a where each micro scan corresponding to a specific ion mobility and RT value has two dimensions, the m/z dimension 202a and the intensity 203a. In one embodiment of real data, a single full IM scan can consist of hundreds of microscans, each with hundreds of data points (i,j) where i stands for m/z (i=0) or intensity (i=1) and j represents the data point index for that dimension. In another embodiment, the micro scans can also be merged. In yet another embodiment, each micro scan in a full scan can be represented by its IM scan index 201a as a placeholder for the actual ion mobility value. In one implementation, different microscans are converted into a single 3D IM Scan 207a by merging all of the individual microscans in the full IM scan into a single array with three dimensions, m/z dimension 204a, intensity dimension 205a, and IM index dimension 206a. This 3D IM scan is sorted by m/z for fast m/z-based access.

FIG. 2 illustrates a basic calibration process.

In one embodiment, the system and method search the reference peptides which can be spiked into the sample and their corresponding decoys in a first step 200. A person with ordinary skill in the art will understand that these reference peptides can be any set of pre-defined reference peptides suitable under the given circumstances. For each reference peptide as available in the library of pre-recorded data, a coordinate or window of interest in the 3 dimensions m/z, IM and RT is retrieved and that window of interest is then searched in the actual data to be analysed.

Then for each peptide in the set determined in the first step, it considers the full range in the RT dimension as the RT window. For each RT position, it accesses the 3D IM scan 207a for building an ion trace in the second step 201.

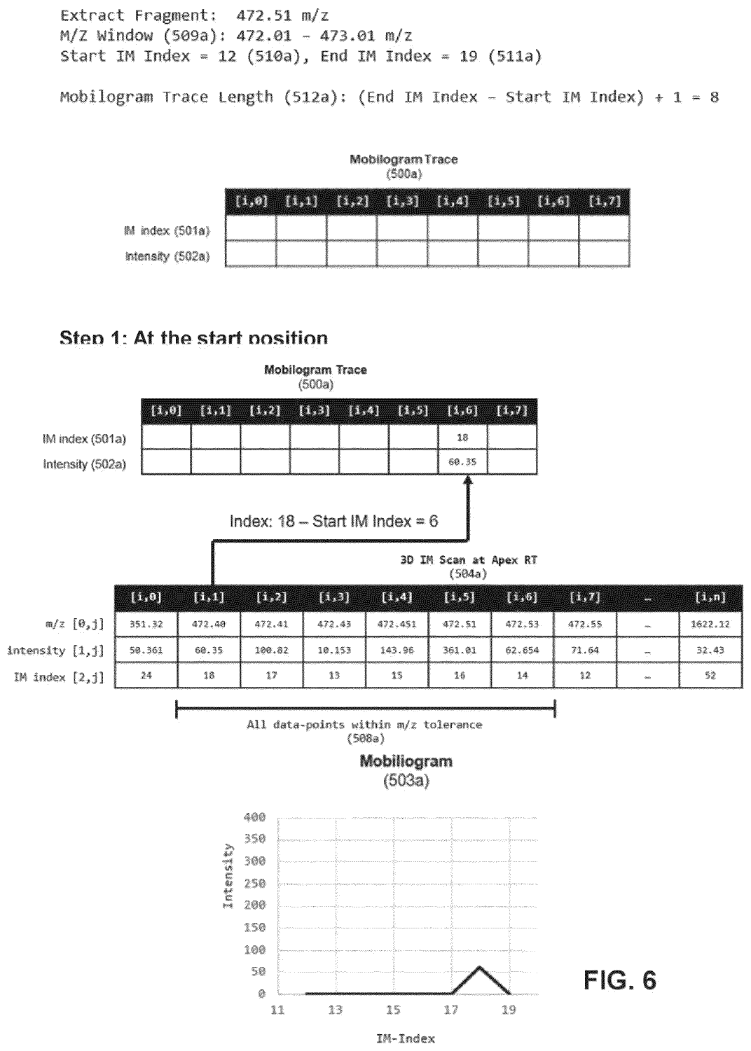

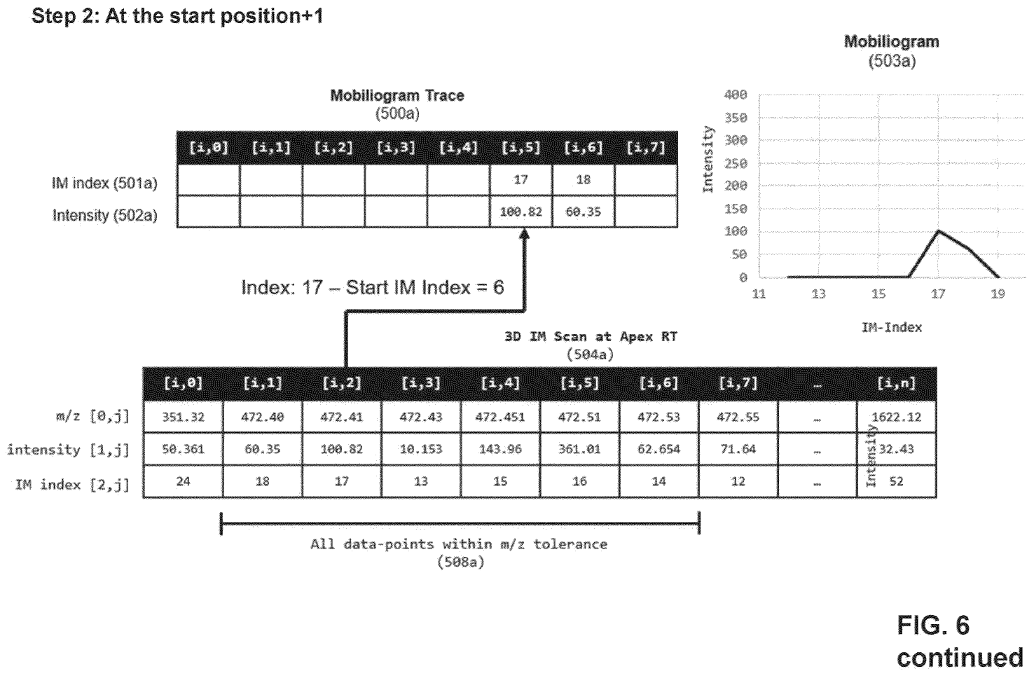

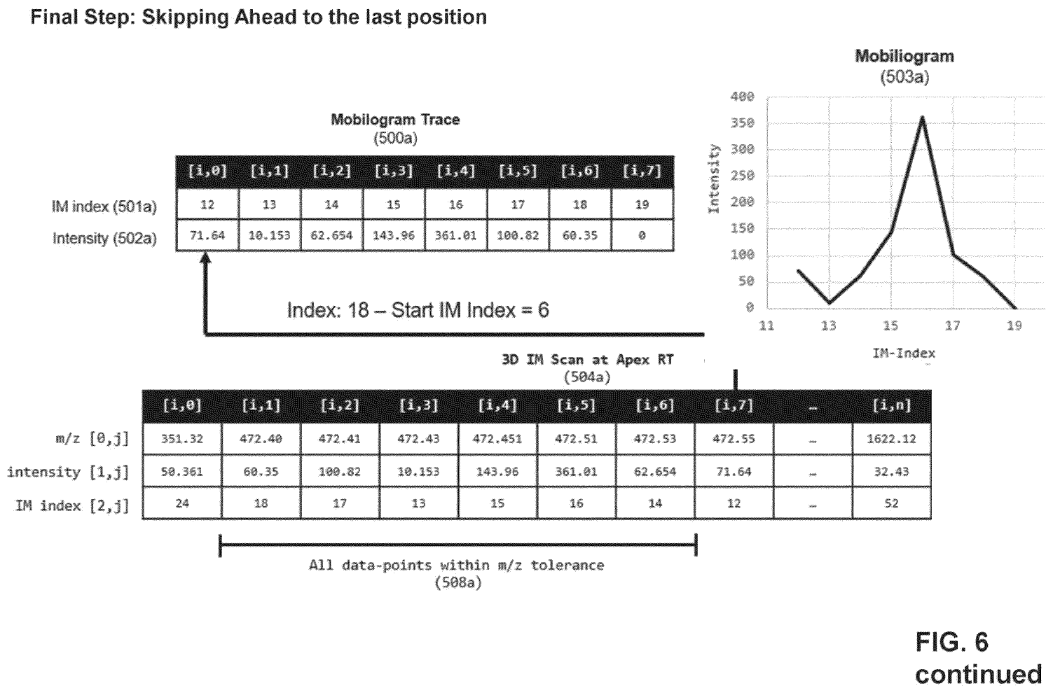

For each 3D IM scan 207a, it condenses the intensity values that fall within the m/z window of the corresponding compound over the full range in the IM dimension to a single data point. Together they build an ion trace in the RT dimension in the third step 202. This process is performed by using the novel method described in FIG. 6.

Peak detection is done for each ion trace to determine the apex retention time and peak width in the following step 203.

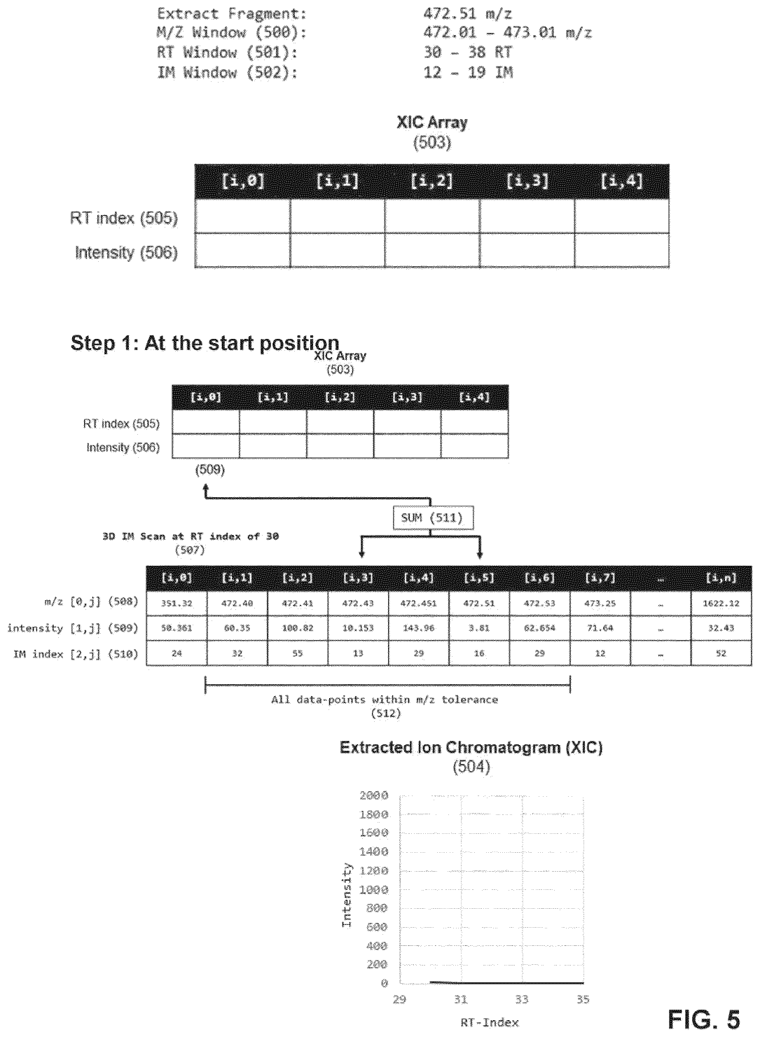

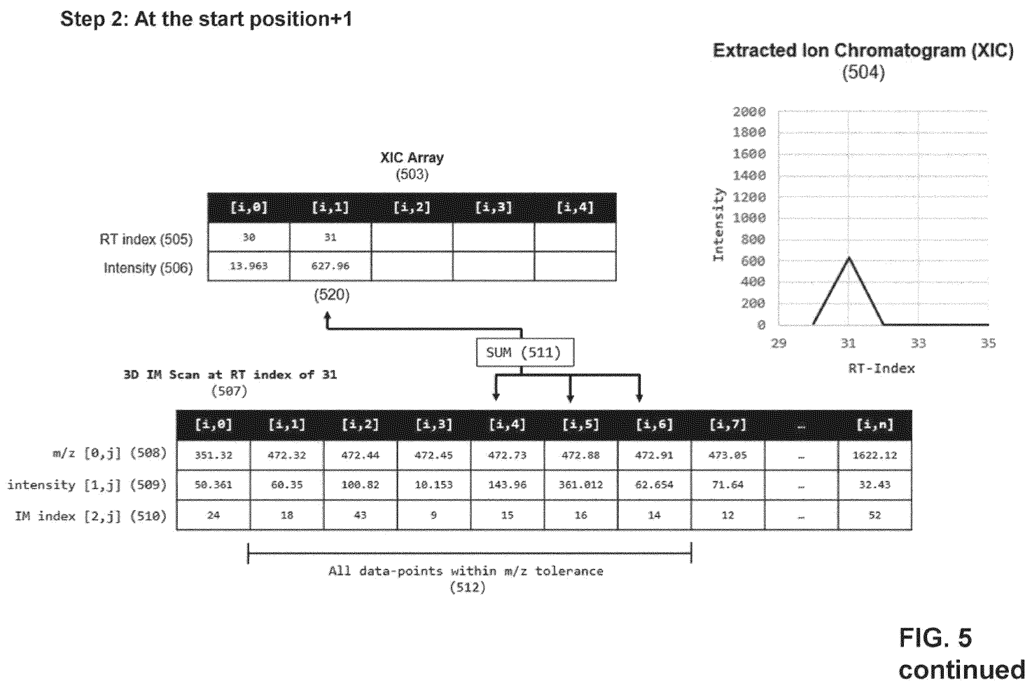

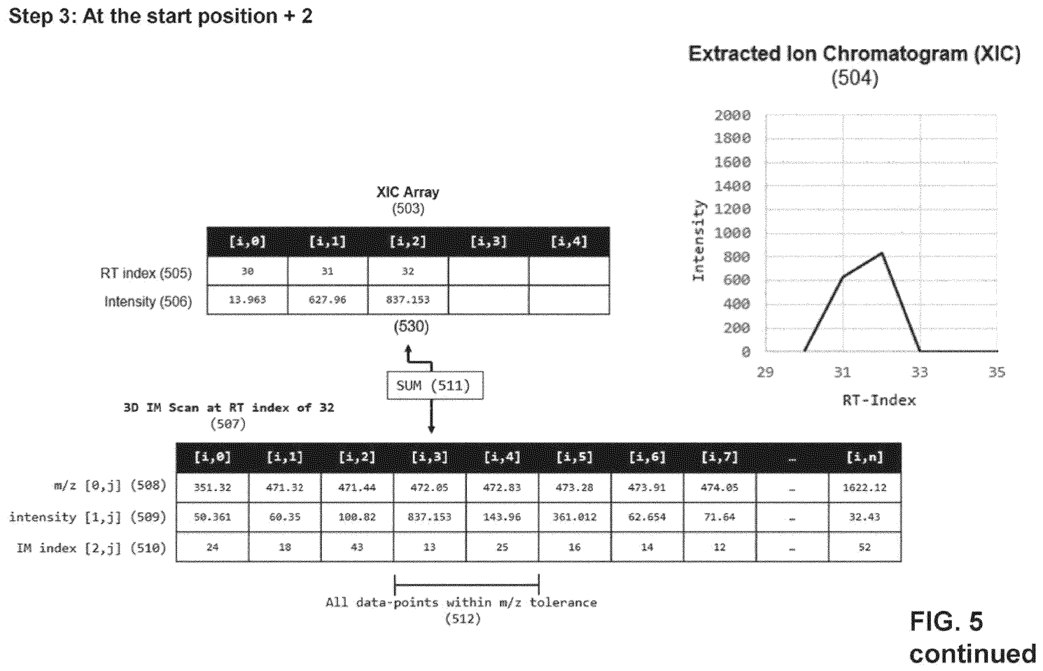

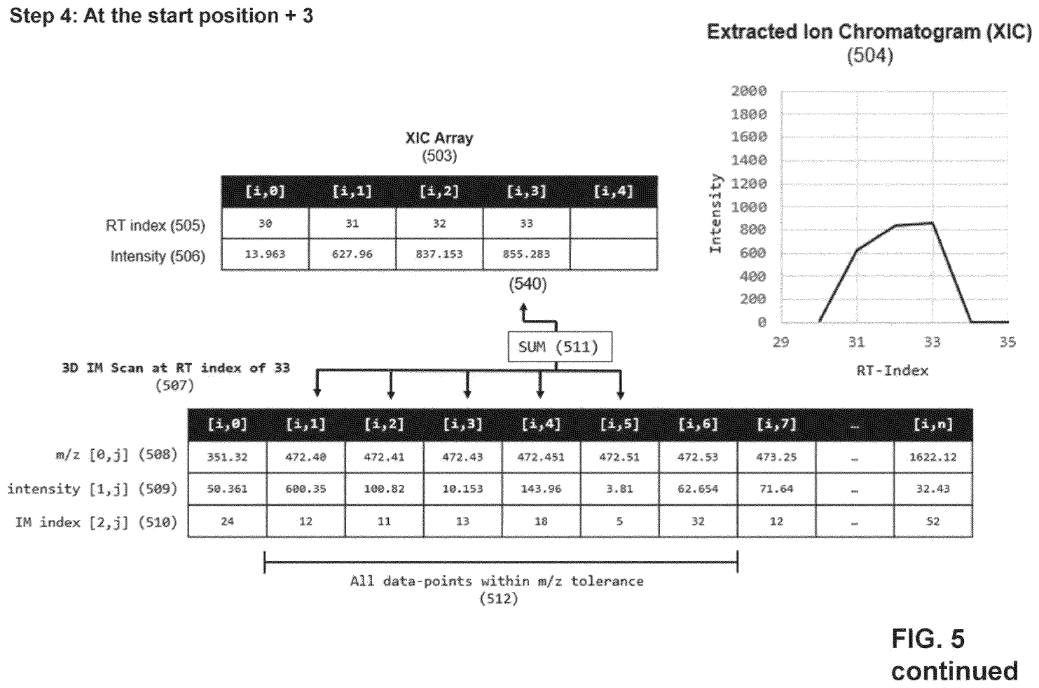

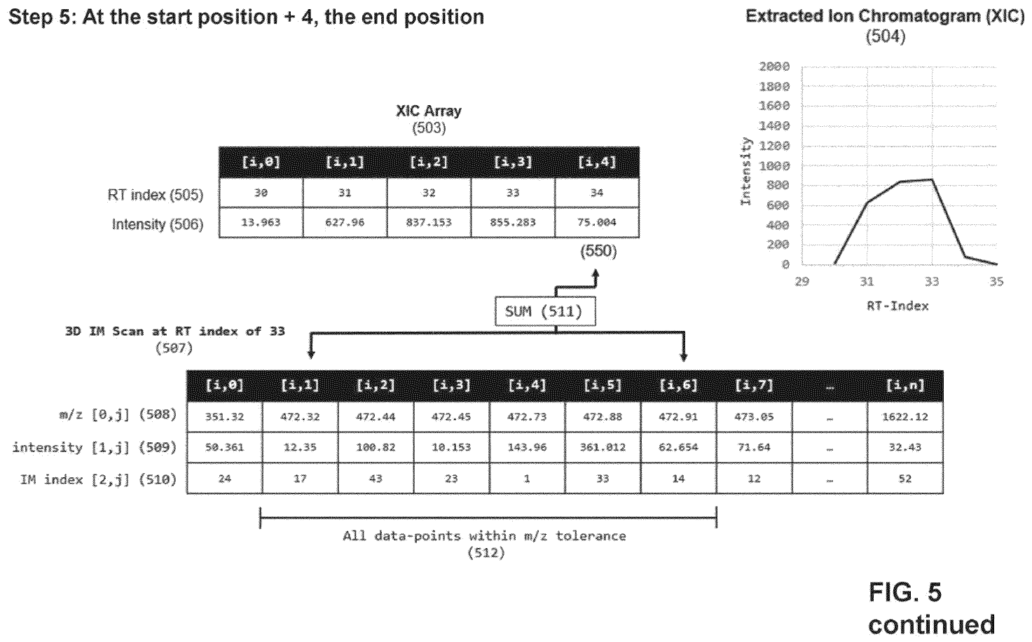

For a 3D IM scan corresponding to the apex RT 103, the ion trace in the IM dimension (mobilogram) is extracted using the same m/z tolerance in the following step 204. Peak detection is done on the mobilogram to find the apex IM at the apex RT which is a good estimation for apex IM 104. Mobilogram is constructed as described in FIG. 5.

Finally, all peptides are scored and identified using a target decoy approach in the following step 205.

If enough peptides are identified (see discriminator 206, yes) then a linear ion mobility regression is created using the apex IM calculated in the following step 207.

If the pre-defined reference peptides were not found (see discriminator 206, no), then random x peptides (e.g. 1000) are selected from the library and this process is performed again as given in step 208.

This can be repeated k times while increasing x until successful.

In another embodiment, the method can directly start with the random selection of library peptides if it was known that the pre-defined reference peptides were not spiked in the sample. A person with ordinary skill in the art will understand that the selection of library peptides does not have to be random and can be done in another manner.

FIG. 3 illustrates an extensive calibration process.

In this implementation, the process is similar to the basic calibration process given in FIG. 2, but utilizes the linear calibrations that were trained in the previous step.

In a first step 300 correspondingly a search list consisting of a larger set of library precursors is created.

In one implementation, the system looks for a larger set of library precursors that are randomly selected. In one embodiment, the set size is either 10% of the library or 10,000 precursors, whichever is larger. In another embodiment, if a library is smaller than 10,000 precursors, the entire library will be used. A person with ordinary skill in the art will understand that any set size can be selected and is not limited to the aforementioned example.

In another embodiment, for each precursor in the set, it uses the RT position and extraction width as per the basic calibration to decide which 3D IM scans to consider for building an ion trace in the following step 301.

For each 3D IM scan, the intensity values that fall within a m/z and IM tolerance to a single data point are condensed. Together they build an ion trace in the RT dimension in the following step 302. This process is performed using our novel method as described in FIG. 5. The IM tolerance is determined based on the IM position and extraction width determined using the linear IM calibration from the previous step.

Peak detection is done for each ion trace to determine the apex retention time in the step 303 that follows.

For a 3D IM scan corresponding to the apex RT 103, the ion trace is extracted in the IM dimension (mobilogram) using the same m/z and IM tolerance as illustrated in step 304. This is done again using the method described in FIG. 5.

Finally, all peptides are scored and identified using a target decoy approach which has been previously described in the context of FIG. 2 as represented in step 305.

If enough peptides are identified (see discriminator 306, yes) then a non-linear ion mobility regression is created using the apex IM calculated in step 307. This regression will be used in the main analysis to calculate high precision IM position and extraction width. A non-linear RT and m/z regression is also created during this step.

If the pre-defined reference peptides were not found (see discriminator 306, no), then a search list consisting of new larger set of library precursors is selected from the library and this process is performed again as given in step 308.

FIG. 4 illustrates the main analysis pipeline.

In one implementation, during the main analysis, the non-linear regressions created during the extensive calibration process are used. In this implementation, all of the precursors in the library and decoys are considered. For each precursor in the set, it uses the RT position and extraction width as per the non-linear calibration from the previous step to decide which 3D IM scans to consider for building an ion trace as given in step 401.

For each 3D IM scan, all intensity values that fall within a m/z and IM tolerance to a single data point are condensed. Together they build an ion trace in the RT dimension as summarised in step 402 in FIG. 5. The IM tolerance is determined based on the IM position and extraction width determined using the non-linear IM calibration from the previous step. In another embodiment, the extraction width is determined using linear IM calibration.

Peak detection is done for each ion trace to determine the apex retention time and peak width in step 403.

For a 3D IM scan array corresponding to the apex RT, the ion trace in the IM dimension (mobilogram) is extracted using the same m/z and IM tolerance as illustrated in step 404. Finally, all peptides are scored and identified using a target decoy approach which has been previously described as represented in step 405.

FIG. 5 illustrates a method to construct an ion trace using an example. Similar to the other implementations and examples mentioned in this disclosure, this example is intended to be non-limiting.

In one implementation, for each full IM scan at a given RT, a 3D IM scan 207a is created. An ion trace is built for a precursor fragment with an expected m/z window 500, RT window 501, and IM window 502. This same process is also applicable to making an ion trace for different precursor isotopes. The 3D IM scans 503 are processed over the RT window. For each 3D IM scan, it will first find the starting position corresponding to the lower bound of the m/z window using a binary search in the m/z dimension. Then it will iterate through each position in the array while the m/z value 508 is still within the tolerance. For each position it iterates, it will sum up all intensity values where the IM index is also within the expected IM window 502. The final sum will be the single data point for that fragment ion at the current RT position 509, 520, 530, 540, 550, respectively for the steps 1-5. Tracing such data points over the entire range of the RT window 501 creates the extracted ion chromatogram 504. Here we describe the first two steps in more depth as an example. In step 1, we start with 3D IM scan at RT index of 30 (reference numeral 507) which is the starting point in RT dimension. In the XIC array, we fill the RT index 505 at position j=0 to be 30. To fill out the intensity 506 at position j=0, we go through the 3D IM scan to find all data points that satisfy the bounds set by the m/z window 500 and IM window 502 and sum them up. This corresponds to data points at j=3 and j=5 in the intensity row 509 of the 3D IM scan. These two data points are summed to fill out the intensity position. In step 2, we increment to the next RT index of 31 (reference numeral 507 in FIG. 5 step 2). In the XIC array, we fill the RT index 505 at position j=1 to be 31. To fill out the intensity at position j=1 506, we look up the 3D IM scan corresponding to this RT index and find all data points that satisfy the bounds set by the m/z window 500 and IM window 502 and sum them up. This corresponds to data points at j=4, 5, 6 and summing them up gives us 627.96.

FIG. 6 illustrates a method to construct a mobilogram 503a using an example. Similar to the other implementations and examples mentioned in this disclosure, this example is intended to be non-limiting.

In one implementation, for each full IM scan at a given RT, a 3D IM scan 207a is created. A mobilogram trace 500a is built for a precursor fragment at its apex RT with an expected m/z window 509a, and IM window with start IM index 510a and end IM index 511a. The trace is a 2-dimensional array (i, j) where i represents the ion mobility (i=1) or intensity dimension (i=2) and j represents the position in the array of length equal to the IM window length 512a. It first finds the starting position corresponding to the lower bound of the m/z window using a binary search in the m/z dimension. Then it iterates through each position in the 3D IM scan while the m/z is still within the tolerance. For each position it iterates where the IM index is also within the expected IM window, it fills the j-th position for ion mobility index and intensity in the mobilogram array where i=current IM index—start IM Index 504a. Iterating through all the positions while the m/z is still within the tolerance creates the mobilogram traces 500a, 503a.

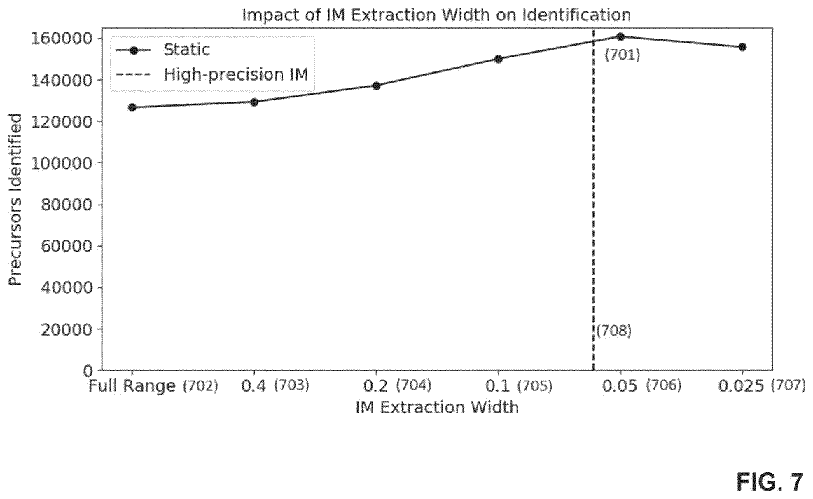

FIG. 7 illustrates an optimal extraction width 701 for each set of experimental data based on the performance of the instrument and quality of the library.

In one implementation, full range extraction 702 is used in addition to different static extraction windows (703, 704, 705, 706, 707) to analyze this dataset. High-precision ion mobility workflow described here automatically finds a near optimal solution 708. High-precision IM ensures that optimal IM extraction width (dashed line, right panel) is automatically used for data analysis as shown here with a 400 ng Hela sample without having to always discover the best extraction width for a new dataset.

A person with ordinary skill in the art will understand that the inventive method and system described in this disclosure can be applied to any intrinsic property of a peptide precursor ion that can be predicted beforehand. Furthermore, while it was only described here for DIA data, it applies for targeted analysis of any mass spec data, e.g. Parallel Reaction Monitoring (PRM). A person skilled in the art will also understand that the present system and method can be extended or applied to any additional dimension of separation.

While certain aspects of the present invention have been particularly shown and described with reference to exemplary embodiments thereof, it will be understood by those of ordinary skill in the art that various changes in form and details may be made therein without departing from the spirit and scope of the present invention as defined by the following claims. It will also be understood that the components of the present disclosure may comprise hardware components or a combination of hardware and software components. The hardware components, methods, and workflows may comprise any suitable tangible components that are structured or arranged to operate as described herein. Some of the hardware components may comprise processing circuitry (e.g., a processor or a group of processors) to perform the operations described herein. The software components may comprise code recorded on tangible computer-readable medium. The processing circuitry may be configured by the software components to perform the described operations. It is therefore desired that the present embodiments be considered in all respects as illustrative and not restrictive.

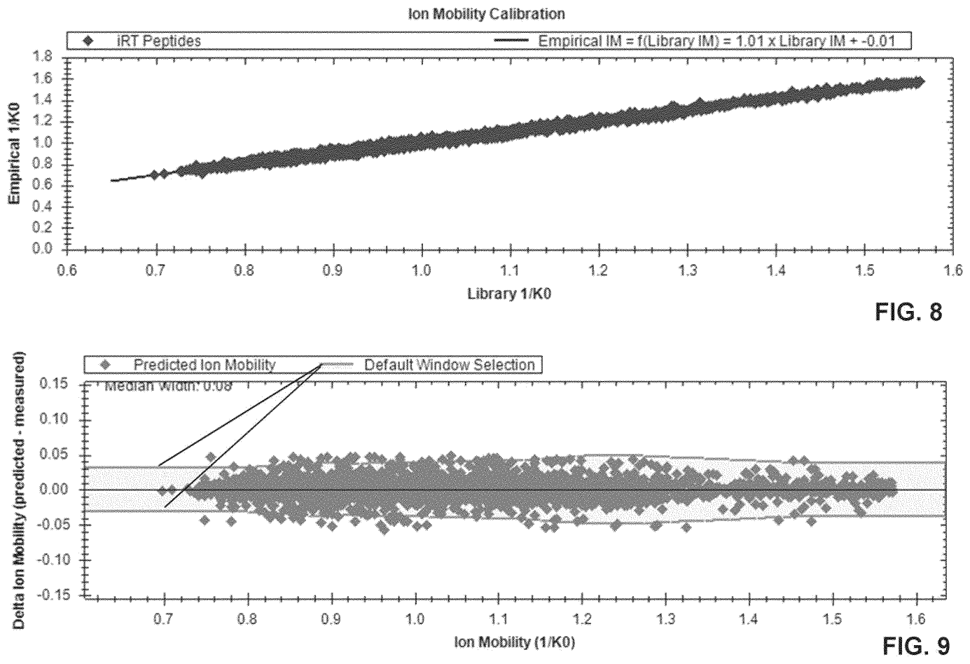

FIG. 8 illustrates an ion mobility calibration (Predicted vs. Empirical) on a sample data set and based on a high number of precursors from a database. In the figure, the x-axis is the predicted ion mobility from a database of reference peptides (expressed in 1/K0 unit) whereas the y-axis is the empirical apex ion mobility calculated on the sample data set as described in the text. In a linear relationship like here, the shift is calculated from the equation shown above the figure. In a non-linear relationship, this is e.g. achieved by using piece-wise regression.

FIG. 9 illustrates an extraction width in ion mobility dimension on a sample data set. In the figure, the x-axis is the empirical apex ion mobility (expressed in 1/K0 unit) versus delta between predicted ion mobility from the database of reference peptides and the empirical apex ion mobility (expressed in 1/K0 unit). The default window selection line indicates the tolerance window calculated based on local quantiles and defines the window that is used around the expected ion mobility of a peptide.

LIST OF REFERENCE SIGNS

| | |
|---|---|
| 100 | ion mobility |
| 101 | retention time |
| 102 | intensity |
| 103 | apex retention time |
| 104 | apex ion mobility |
| 200a | IM micro scan |
| 201a | IM scan index |
| 202a | m/z dimension of 200a |
| 203a | intensity dimension of 200a |
| 204a | m/z dimension of 207a |
| 205a | intensity dimension of 207a |
| 206a | IM index dimension of 207a |
| 207a | 3D IM scan |
| 500 | expected m/z window |
| 500a | mobilogram trace |
| 501 | expected RT window |
| 501a | IM index |
| 502 | expected IM window |
| 502a | intensity |
| 503 | 3D IM scan |
| 503 | mobilogram |
| 503a | mobilogram trace |
| 504 | XIC |
| 504a | 3D IM scan at apex RT |
| 505 | RT index |
| 506 | intensity |
| 507 | 3D IM scan at RT index of 30 |
| 508 | m/z value |
| 508a | data points within m/z tolerance |
| 509 | single data point for that fragment ion at the current RT position |
| 509a | expected m/z window |
| 510 | IM index value |
| 510a | start IM index |
| 511 | sum |
| 511a | end IM index |
| 512 | data points within m/z tolerance |
| 512a | mobilogram trace length |
| 520 | single data point for that fragment ion at the current RT position |
| 530 | single data point for that fragment ion at the current RT position |
| 540 | single data point for that fragment ion at the current RT position |
| 550 | single data point for that fragment ion at the current RT position |
| 701 | optimal extraction width |
| 702 | full range extraction |
| 703-707 | different static extraction windows |
| 708 | high precision ion mobility workflow extraction width value |
| IM | ion mobility |
| RT | retention time |
| XIC | extracted ion chromatograms |

1. Method for the targeted peptide precursor identification from sample mass spectroscopic intensity data acquired as a function of mass to charge ratio, of retention time as well as of ion mobility,
using a database of reference peptide precursor data for retrieval of a region of interest for at least three reference peptide precursors in the mass to charge ratio, the retention time as well as in the ion mobility dimension,
wherein for peptide precursor identification from the sample mass spectroscopic intensity data,
in a first step for at least three reference peptide precursors, from the database of reference peptide precursor data, said sample mass spectroscopic intensity data is analysed in the respective reference peptide precursor region of interest of mass to charge ratio, retention time as well as ion mobility dimension, and from that analysis empirically an adjusted center in the ion mobility dimension for each reference peptide precursor is determined and an ion mobility extraction width window in the ion mobility dimension, is determined, and wherein
in a second step for the identification of further peptide precursors from said sample mass spectroscopic intensity data, said empirically determined ion mobility extraction width window in the ion mobility dimension, is used.

2. Method according to claim 1, wherein for the analysis in the first and/or the second step, for a given retention time the data are merged into a single array with three dimensions, mass to charge ratio dimension, intensity dimension, and ion mobility index dimension, sorted by mass to charge ratio.

3. Method according to claim 2, wherein in said first step for each reference peptide precursor the analysis
considers a range in the retention time dimension as a retention time window,
and for each retention time value in that retention time window, it accesses said single array for that retention time for building a first ion trace for that reference peptide precursor,
in that the intensity values that fall within a mass to charge ratio window of the corresponding reference peptide are summed up over the full range in the ion mobility dimension to a single data point for that retention time value, these single data points as a function of the retention time value building together said first ion trace in the retention time dimension,
followed by peak detection in the first ion trace to determine the apex retention time.

4. Method according to claim 2, wherein the building of the first ion trace and the determination of the apex retention time for that reference peptide precursor is
followed by extraction of a second ion trace at said trace apex retention time by accessing said single array for that trace apex retention time for building a second ion trace for that reference peptide precursor,
in that the intensity values that fall within a mass to charge ratio window of the corresponding reference peptide are extracted and represented as a function of the ion mobility dimension, building together said second ion trace in the ion mobility dimension,
followed by peak detection in the second ion trace to determine the apex ion mobility value and the peak width for that reference peptide precursor.

5. Method according to claim 4, wherein the peak detection in the second ion trace is followed by a non-linear or linear ion mobility regression using the apex ion mobility values and the peak widths of the at least three reference peptide precursors to determine said adjusted center in the ion mobility dimension for each reference peptide precursor and to determine said window in the ion mobility dimension.

6. Method according to claim 3, wherein said first step is carried out for a first set of at least 3, or at least 5, or at least 7, or at least 10, or at least 100, or at least 1000 reference peptide precursors considering the full range in the retention time dimension as retention time window for building the first ion trace, and wherein said first step is carried out a second time with a larger set of reference peptide precursors than the first time, or at least 10, or at least 100 or at least 1000, or at least 2000, or at least 5000 reference peptide precursors, and wherein a retention time window is used for building the first ion trace based on the peak width in the retention time dimensions determined in the first run of the first step.

7. Method according to claim 1, wherein using a database of reference peptide precursor data for a region of interest for at least 5, or at least 7, or at least 10, or at least 100, or at least 500, or at least 1000 reference peptide precursors in the mass to charge ratio, the retention time as well as in the ion mobility dimension is retrieved for carrying out the first step.

8. Method according to claim 1, wherein in said second step after the determination of the precursors in the data, for each precursor the analysis
considers a range in the retention time dimension as a retention time window,
and for each retention time value in that retention time window, it accesses said single array for that retention time for building a first ion trace for that precursor,
in that the intensity values that fall within a mass to charge ratio window of the corresponding precursor are summed up over the full range in the ion mobility dimension to a single data point for that retention time value,
these single data points as a function of the retention time value building together said first ion trace in the retention time dimension,
followed by peak detection in the first ion trace to determine the apex retention time.

9. Method according to claim 8, wherein the step of peak detection in the first ion trace to determine the apex retention time is followed by extraction of a second ion trace at said trace apex retention time by accessing said single array for that trace apex retention time for building a second ion trace for that precursor,
in that the intensity values that fall within a mass to charge ratio window of the corresponding precursor are extracted and represented as a function of the ion mobility dimension, building together said second ion trace in the ion mobility dimension,
followed by peak detection in the second ion trace to determine the apex ion mobility value and the peak width for that precursor for scoring and/or identification of the precursor.

10. Method according to claim 1, wherein in the first step also in the mass to charge ratio dimension and/or in the retention time dimension an extraction width window in the respective dimension, is determined, and in the second step for the identification of further peptide precursors from said sample mass spectroscopic intensity data, said empirically determined extraction width window in the respective dimension as a variable function of the respective dimension is used.

11. Method according to claim 1, wherein the sample mass spectroscopic intensity data acquired as a function of mass to charge ratio, of retention time as well as of ion mobility are determined using an LC tandem mass spectroscopy method, is used.

12. Method according to claim 1, wherein the sample mass spectroscopic intensity data acquired as a function of mass to charge ratio, of retention time as well as of ion mobility are determined from a biological sample in a data independent manner on the complete mass range, and through the entire chromatography, disregarding of the content of the sample.

13. Method according to claim 1, wherein at least one, or at least 2, or at least 5, or at least 7 reference peptide precursors are based on a corresponding number of proteins or peptides spiked into the sample to be analysed prior to analysis.

14. Method for the generation of a peptide precursor database, wherein at least one or for the majority or for each peptide precursor in the database based on empirical measurements an associated ion mobility extraction width window is determined and stored in the database and using a method according to claim 1.

15. A computer program product, whose contents include a program with instructions being executed on the processor so as to control a device for chemical analysis using a method according to claim 1.

16. Method according to claim 1, wherein in a first step for at least three reference peptide precursors, or for all reference peptide precursors from the database of reference peptide precursor data, said sample mass spectroscopic intensity data is analysed in the respective reference peptide precursor region of interest of mass to charge ratio, retention time as well as ion mobility dimension, and from that analysis empirically an adjusted center in the ion mobility dimension for each reference peptide precursor is determined and an ion mobility extraction width window in the ion mobility dimension, as a variable function of the ion mobility dimension, is determined, and wherein
in a second step for the identification of further peptide precursors from said sample mass spectroscopic intensity data, said empirically determined ion mobility extraction width window in the ion mobility dimension, as a variable function of the ion mobility dimension is used.

17. Method according to claim 1, wherein in said first step for each reference peptide precursor the analysis
considers a range in the retention time dimension as a retention time window, or the full retention time dimension,
and for each retention time value in that retention time window, it accesses said single array for that retention time for building a first ion trace for that reference peptide precursor,
in that the intensity values that fall within a mass to charge ratio window of the corresponding reference peptide are summed up over the full range in the ion mobility dimension to a single data point for that retention time value,
these single data points as a function of the retention time value building together said first ion trace in the retention time dimension,
followed by peak detection in the first ion trace to determine the apex retention time and also the peak width in the retention time dimension for that reference peptide precursor.

18. Method according to claim 2, wherein the building of the first ion trace and the determination of the apex retention time and also the peak width in the retention time dimension for that reference peptide precursor is
followed by extraction of a second ion trace at said trace apex retention time by accessing said single array for that trace apex retention time for building a second ion trace for that reference peptide precursor,
in that the intensity values that fall within a mass to charge ratio window of the corresponding reference peptide are extracted and represented as a function of the ion mobility dimension, building together said second ion trace in the ion mobility dimension,
followed by peak detection in the second ion trace to determine the apex ion mobility value and the peak width for that reference peptide precursor.

19. Method according to claim 1, wherein in said second step after the determination of the precursors in the data, for each precursor the analysis
considers a range in the retention time dimension as a retention time window, determined based on an analysis of reference peptide precursors,
and for each retention time value in that retention time window, it accesses said single array for that retention time for building a first ion trace for that precursor,
in that the intensity values that fall within a mass to charge ratio window of the corresponding precursor are summed up over the full range in the ion mobility dimension to a single data point for that retention time value,
these single data points as a function of the retention time value building together said first ion trace in the retention time dimension,
followed by peak detection in the first ion trace to determine the apex retention time and also the peak width in the retention time dimension for that precursor.

20. Method according to claim 8, wherein the step of peak detection in the first ion trace to determine the apex retention time and also the peak width in the retention time dimension for that precursor is followed by extraction of a second ion trace at said trace apex retention time by accessing said single array for that trace apex retention time for building a second ion trace for that precursor,
in that the intensity values that fall within a mass to charge ratio window of the corresponding precursor are extracted and represented as a function of the ion mobility dimension, building together said second ion trace in the ion mobility dimension,
followed by peak detection in the second ion trace to determine the apex ion mobility value and the peak width for that precursor for scoring and/or identification of the precursor.

21. Method according to claim 1, wherein in the first step also in the mass to charge ratio dimension and/or in the retention time dimension an extraction width window in the respective dimension, as a variable function of the respective dimension, is determined, and in the second step for the identification of further peptide precursors from said sample mass spectroscopic intensity data, said empirically determined extraction width window in the respective dimension as a variable function of the respective dimension is used.

22. Method according to claim 1, wherein the sample mass spectroscopic intensity data acquired as a function of mass to charge ratio (m/z), of retention time as well as of ion mobility are determined using an LC tandem mass spectroscopy method, selected from the group of LC-MRM or LC-SWATH is used.

23. Method according to claim 1, wherein the sample mass spectroscopic intensity data acquired as a function of mass to charge ratio (m/z), of retention time as well as of ion mobility are determined from a biological sample in a data independent manner on the mass rang, 200-2000 Thomson, and through the entire chromatography, disregarding of the content of the sample.

24. Method according to claim 14, wherein for the majority or for each peptide precursor in the database based on empirical measurements an associated ion mobility extraction width window is determined and stored in the database determined from sample mass spectroscopic intensity data acquired as a function of mass to charge ratio, of retention time as well as of ion mobility.

25. A computer program product according to claim 15, on or comprising a tangible computer-readable storage medium.
